(19) United States
(12) Patent Application Publication
(10) Pub. No.: US 2011/0002859 A1
(43) Pub. Date: Jan. 6, 2011

(54) CHEWING GUM CONTAINING EUCALYPTUS EXTRACT

(75) Inventors: Yuuichi Maeda, Saitama-shi (JP); Takahito Takase, Asaka-shi (JP); Atsushi Narise, Kawasaki-shi (JP); Kenji Osawa, Saitama-shi (JP)

Correspondence Address: MILES & STOCKBRIDGE PC, 1751 PINNACLE DRIVE, SUITE 500, MCLEAN, VA, 22102-3833, US

(73) Assignee: LOTTE CO., LTD., Tokyo (JP)

(21) Appl. No.: 12/920,734

(22) PCT Filed: Feb. 17, 2009
(86) PCT No.: PCT/JP2009/053120

§ 371 Date: Sep. 2, 2010

(30) Foreign Application Priority Data
Mar. 3, 2008 (JP) 2008-052267

(51) Int. Cl. A61K 8/97; A61K 9/68; A61P 43/00; A61P 31/04; A61Q 11/00

(52) U.S. Cl. 424/48

(57) ABSTRACT

It is an object of the present invention to provide a chewing gum, by which an effect of preventing periodontal disease is obtained through mastication without performance required for a chewing gum being impaired, such as taste, appearance, and physical properties and storage stability as a product. A chewing gum containing Eucalyptus extract of the present invention is characterized by containing at least one kind of macrocarpal A, macrocarpal B, and macrocarpal C, contents of the macrocarpal A, the macrocarpal B, and the macrocarpal C being 0.001 to 0.0045% by weight, 0.003 to 0.0045% by weight, and 0.0015 to 0.0045% by weight with respect to a total weight of the chewing gum, respectively.

### TECHNICAL FIELD

The present invention relates to a chewing gum containing Eucalyptus extract. Specifically, the present invention relates to a chewing gum containing Eucalyptus extract, in which an effect of preventing periodontal disease is obtained through mastication without performance required for a chewing gum being impaired, such as taste, appearance, and physical properties and storage stability as a product.

### BACKGROUND ART

It has been found that caries and periodontal disease are two major intraoral diseases, both of which are infections caused by a specific bacterium. The periodontal disease is multifactorial disease due to an agent, an environment, and a host. Of those, it is bacteria that play the most etiologic role. Recent researches have reported some pathogenic bacteria, pathogenicity factors thereof, and the like.

Of pathogenic bacteria, Porphyromonas gingivalis has been dominantly regarded as a pathogenic bacterium causing adult periodontitis, which is the most common periodontal disease. This is because Porphyromonas gingivalis is frequently isolated from a periodontal pocket of a patient suffering from periodontitis and has strong pathogenicity factors such as collagenase, immunoglobulinase, fibroblast growth inhibitory activity, and volatile sulfide. In addition, Prevotella intermedia, Prevotella melaninogenica, Capnocytophaga ochracea, and the like have been clearly identified as possible periodontopathic bacteria.

In order to prevent periodontal disease, it is important to inhibit the growth of pathogenic bacteria in foci and to suppress pathogenicity factors of pathogenic bacteria. From the viewpoint of suppressing the pathogenic bacteria, an antibacterial substance effective against bacteria is effectively applied. Conventionally, antibiotics such as tetracycline and minocycline have been used. However, those antibiotics are not suitable for routine applications in spite of having potent activities because they generate drug-resistant bacteria and have adverse effects, for example.

So in recent years, an attempt has been made that a natural product, in particular, Eucalyptus extract is used for prevention and treatment of periodontal disease. It has been known that an essential oil component of Eucalyptus (Eucalyptus oil) and cineole (eucalyptol) as its main component enhance an effect as a preservative and an activity of an antibacterial substance (Japanese Patent Application Laid-Open No. 62-289511). However, those Eucalyptus oil and cineole themselves have weak antibacterial activities against periodontopathic bacteria, and besides have strong distinct odor. Therefore, there is a problem that, at the time of use in oral cavity, the application and the addition amount are restricted to a considerable extent.

Japanese Patent Application Laid-Open No. 5-306252 relates to macrocarpals extracted from Eucalyptus and pharmaceutically acceptable salts thereof, and particularly describes that macrocarpal A has an antibacterial activity. In addition, there is also described that macrocarpals have aldose reductase inhibitory activity, and are valuable as a medicament for therapeutic treatment of diabetes complications such as cataract, neuropathy, retinopathy, and nephropathy. However, Japanese Patent Application Laid-Open No. 5-306252 discloses none of periodontal disease as an oral disease and periodontopathic bacteria in bacteria on which tests were performed.

Japanese Patent No. 2804232 discloses an anticarious and antiperiodontopathic agent containing a phloroglucinol derivative extracted from Eucalyptus as an active ingredient, and an oral composition containing the anticarious and antiperiodontopathic agent. Further, macrocarpal A, macrocarpal B, macrocarpal C, and macrocarpal D are described as specific examples of the phloroglucinol derivative. There is described that when the anticarious and antiperiodontopathic agent is compounded into foods or sanitary materials, the compounding proportion is preferably 0.001 to 10% by weight. An example of applications thereof includes a chewing gum. In addition, there is described a prescription of a chewing gum containing 0.2% of macrocarpal A as an application example.

Japanese Patent No. 3547835 discloses an oral composition containing macrocarpal A, macrocarpal B, macrocarpal C, macrocarpal D, and the like as an agent for preventing and treating throat inflammation and hemolysin. Further, an example of applications thereof includes a chewing gum, and there is described that when the oral composition is compounded into foods, the compounding proportion is preferably 0.001 to 10% by weight. In addition, as an application example, there is described a prescription of a chewing gum containing 0.2% of Eucalyptus extract which is produced in examples. However, no description is given as to periodontal disease.

On the other hand, when the amount of Eucalyptus extract increases beyond a certain amount, adverse effects appear as follows: distinct odor becomes strong, thereby adversely affecting the product taste; the gum surface changes in color, and hence the appearance becomes worse; drying of a chewing gum in a production step becomes difficult, and thus the shape becomes worse; and besides, the storage stability becomes poor due to moisture absorption even after its production.

The content of 0.2% of macrocarpal A in a chewing gum described in an application example of Japanese Patent No. 2804232, and the content of 0.2% of Eucalyptus extract described in an application example of Japanese Patent No. 3547835 are clearly high as the content in a chewing gum product, and hence the above-mentioned problems are involved, and neither of the substances are practical.

Accordingly, it is considered that the market highly needs a chewing gum, in which macrocarpals having antibacterial activities are incorporated, and which can prevent periodontal disease through a simple action of mastication without characteristics of a conventional chewing gum being impaired.

### DISCLOSURE OF THE INVENTION

The present invention has been made to solve the above-mentioned problems. It is an object of the present invention to provide a chewing gum with which periodontal disease may be prevented, and besides, taste is not affected, appearance of the chewing gum is not impaired, production of the chewing gum is not adversely affected, and storage stability is not impaired even after production.

A chewing gum containing Eucalyptus extract as one aspect of the present invention is characterized by containing macrocarpal A in an amount of 0.001 to 0.0045% by weight with respect to the total weight of the chewing gum.

Further, a chewing gum containing Eucalyptus extract as another aspect of the present invention is characterized by containing macrocarpal B in an amount of 0.003 to 0.0045% by weight with respect to the total weight of the chewing gum.

Still further, a chewing gum containing Eucalyptus extract as still another aspect of the present invention is characterized by containing macrocarpal C in an amount of 0.0015 to 0.0045% by weight with respect to the total weight of the chewing gum.

In addition, in the present invention, as described above, the effect of preventing periodontal disease may be sufficiently obtained by compounding any one kind of the macrocarpal A, B, and C. In order to further enhance the effect, the above aspects may be combined. In other words, as another aspect, the chewing gum containing Eucalyptus extract of the present invention is characterized by containing: at least two kinds of the macrocarpal A, macrocarpal B, and macrocarpal C; and the macrocarpal A, macrocarpal B, and macrocarpal C in an amount of 0.001 to 0.0045% by weight, 0.003 to 0.0045% by weight, and 0.0015 to 0.0045% by weight with respect to the total weight of the chewing gum, respectively.

The macrocarpals A, B, and C to be compounded into the chewing gum of the present invention are phloroglucinol derivatives, and compounds each represented by the following formulae. Those macrocarpals may be extracted from Eucalyptus leaves according to a method described in Japanese Patent No 3547835.

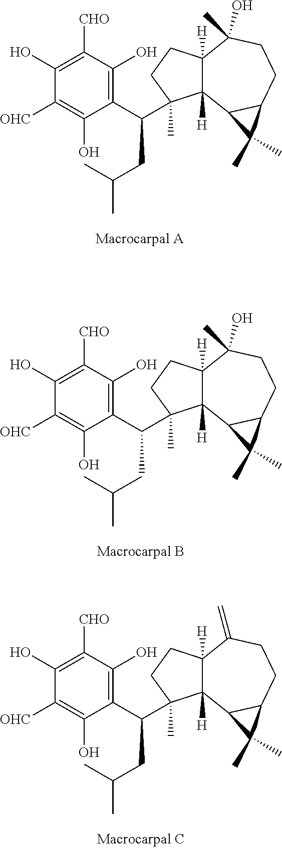

As mentioned above, in one aspect of the present invention, the macrocarpal A is contained in an amount of 0.001 to 0.0045% by weight with respect to the total weight of the chewing gum. When the content of the macrocarpal A is less than 0.001% by weight, a sufficient antibacterial effect against periodontopathic bacteria is not obtained. On the other hand, when the content of the macrocarpal A exceeds 0.0045% by weight, taste becomes poor, appearance as a product becomes worse, and besides, storage stability after production becomes poor. Further, in another aspect of the present invention, the macrocarpal B is contained in an amount of 0.003 to 0.0045% by weight with respect to the total weight of the chewing gum. When the content of the macrocarpal B is less than 0.003% by weight, a sufficient antibacterial effect against periodontopathic bacteria is not obtained. On the other hand, when the content of the macrocarpal B exceeds 0.0045% by weight, taste becomes poor, appearance as a product becomes worse, and besides, storage stability after production becomes poor. Still further, in still another aspect of the present invention, the macrocarpal C is contained in an amount of 0.0015 to 0.0045% by weight with respect to the total weight of the chewing gum. When the content of the macrocarpal C is less than 0.0015% by weight, a sufficient antibacterial effect against periodontopathic bacteria is not obtained. On the other hand, when the content of the macrocarpal C exceeds 0.0045% by weight, taste becomes poor, appearance as a product becomes worse, and besides, storage stability after production becomes poor.

In addition, in the present invention, those macrocarpals A, B, and C may be used in combination.

According to the chewing gum of the present invention, a remarkable effect as described below may be obtained.

1) The growth of periodontopathic bacteria may be inhibited, and hence periodontal disease may be prevented while mastication of a chewing gum is being enjoyed through a simple action of masticating the chewing gum.

2) There is no odor characteristic with Eucalyptus extract, and hence the taste of the chewing gum is not impaired.

3) Eucalyptus extract neither mottles the color of the surface of the chewing gum as a product nor makes the surface irregular, and hence its appearance is not impaired.

4) In the production step of the chewing gum, physical properties of the chewing gum are not changed, and hence the shape as the chewing gum is not impaired.

5) No time-dependent change due to moisture absorption and the like occurs after production, and hence storage stability is not impaired.

### BEST MODES FOR CARRYING OUT THE INVENTION

A chewing gum as stated in the present invention refers to a product obtained by: mixing a gum base with a sweet ingredient, an acidulous ingredient, a flavor, and the like, as required; and further subjecting the resultant to forming and sugar-coating, as required.

Examples of the chewing gum include a sugar-coated gum, a plate gum, a block gum, a tube gum, and a stick gum.

The chewing gum of the present invention may be obtained by compounding at least one kind of macrocarpal A, macrocarpal B, and macrocarpal C in a conventional chewing gum.

As ingredients of the chewing gum of the present invention, ingredients which have been conventionally used for a chewing gum may be used. Examples thereof include: gum base; high intensity sweeteners such as acesulfame K and aspartame; sweeteners such as xylitol, maltitol, reduction maltose starch syrup, sucralose, and glucose; thickeners such as acacia gum, pectin, and guar gum; and other compounding agents such as flavors, brighteners, and pigments.

The macrocarpal A, macrocarpal B, and macrocarpal C may be obtained as follows.

First, leaves of a plant of the genus Eucalyptus, for example, Eucalyptus were pulverized by any appropriate pulverizing means such as a pulverizer, and essential oil components are preferably removed with a device for steam distillation which is generally used in essential oil extraction. As for the removal of essential oil components, low polar solvents such as n-hexane and petroleum ether may be used to extract at normal temperature.

The thus obtained essential oil extraction residue is extracted by applying at least one solvent of polar solvents such as ethyl acetate, acetone, ethanol, methanol, and water, and the resultant extraction solution is concentrated under reduced pressure to afford an extract. The extract may be further separated and purified by appropriate separation and purification means such as column chromatography, to thereby afford the macrocarpal A, macrocarpal B, and macrocarpal C which are phloroglucinol derivatives in the forms of compounds (see Japanese Patent No. 3547835).

In the present invention, when the macrocarpal A is compounded, the preferable compounding amount of the macrocarpal A is 0.0005 to 0.007% by weight and preferably 0.001 to 0.0045% by weight with respect to the total weight of a chewing gum. Further, when the macrocarpal B is compounded, the preferable compounding amount of the macrocarpal B is 0.0008 to 0.007% by weight and preferably 0.003 to 0.0045% by weight with respect to the total weight of a chewing gum. Still further, when the macrocarpal C is compounded, the preferable compounding amount of the macrocarpal C is 0.0005 to 0.007% by weight and preferably 0.0015 to 0.0045% by weight with respect to the total weight of a chewing gum.

In addition to macrocarpals, the kind and amount of compounding agents, which have been conventionally used, are selected depending on the characteristics of a chewing gum required individually. The compounding agents are compounded and mixed with a mixer or the like, and then formed into a stick gum, a block gum, or the like, whereby a chewing gum is obtained as a final product.

Hereinafter, the present invention is specifically described by way of examples. The examples are illustrative, and the present invention is not limited thereto.

### Example 1

In vitro antibacterial activities of Macrocarpal A, macrocarpal B, and macrocarpal C were evaluated according to the following procedure. First, to 25 ml of a medium, added were 2.5 ml of various pre-cultured periodontopathic bacteria described below, and the whole was mixed well. A sample solution was prepared by diluting in two stages a sample stock solution on a 96-well plate by using a diluent. Within each well of the 96-well plate, 100 μl of the sample solution and 100 μl of a bacterial solution were mixed, and cultivated at 37° C. under an anaerobic condition for 24 to 48 hours. The turbidity (550 nm) of each well was measured with a microplate reader, the growth of the bacterium was assessed, and its minimum inhibitory concentration (MIC) was determined. Table 1 shows the results.

(Used Bacterial Strain)

a: Porphyromonas gingivalis ATCC33277

b: Prevotella intermedia ATCC25611

c: Prevotella melaninogenica ATCC25845

d: Capnocytophaga ochracea ATCC33596

TABLE 1
Macrocarpals and minimum inhibitory
concentrations (MIC) for each bacterial strain

| | MIC (μg/ml) | | | |
|---|---|---|---|---|
| Used bacterial strain | a | b | c | d |
| Macrocarpal A | 0.195 | 0.195 | 0.78 | 0.39 |
| Macrocarpal B | 0.78 | 1.58 | 3.13 | 1.56 |
| Macrocarpal C | 0.195 | 0.39 | 1.58 | 0.39 |

As shown in Table 1, it was confirmed that the macrocarpal A, B, and C were capable of preventing periodontopathic bacteria from growing up in concentrations of 0.78 μg/ml, 3.13 μg/ml, and 1.58 μg/ml, respectively.

### Example 2

In order to investigate appropriate compounding concentrations of macrocarpal A, B and C in a chewing gum, chewing gums A, B, and C were prepared according to the following procedure. It should be noted that the unit is % by weight.

The specific compounding amounts of the macrocarpal A, macrocarpal B, and macrocarpal C in the following prescriptions are described in Table 2, Table 3, and Table 4, respectively.

<Chewing gum A>

| | |
|---|---|
| Xylitol | 45.0 |
| Maltitol | 33.0 |
| Gum base | 14.0 |
| Flavor | 1.0 |
| Pigment | 0.1 |
| Acacia gum | balance |
| Macrocarpal A | 0.00050 to 0.00552 |
| Total | 100.0 |

<Chewing gum B>

| | |
|---|---|
| Xylitol | 45.0 |
| Maltitol | 33.0 |
| Gum base | 14.0 |
| Flavor | 1.0 |
| Pigment | 0.1 |
| Acacia gum | balance |
| Macrocarpal B | 0.00120 to 0.00556 |
| Total | 100.0 |

<Chewing gum C>

| | |
|---|---|
| Xylitol | 45.0 |
| Maltitol | 33.0 |
| Gum base | 14.0 |
| Flavor | 1.0 |
| Pigment | 0.1 |
| Acacia gum | balance |
| Macrocarpal C | 0.00090 to 0.00495 |
| Total | 100.0 |

### Example 3

In order to measure the macrocarpal concentration in saliva at the time of masticating a chewing gum, 3 g of a test chewing gum prepared in Example 2 were masticated for 5 minutes, and all amount of saliva from mastication onset to 5 minutes later were collected. The collected saliva was added with acetonitrile and stirred well to denature a protein, and the solution was adjusted to be 50 ml. The solution was centrifuged, and then the supernatant was filtered with a filter, and the filtrate was taken to be a test solution. Tables 2 to 4 show the results of the determined macrocarpal concentration in saliva.

### Example 4

In order to investigate the antibacterial activity of the macrocarpal in the saliva after the mastication of a chewing gum, the minimum inhibitory concentrations (MIC) obtained from the results of Example 1 and concentrations of macrocarpals in saliva from Example 2 were compared. As for evaluation, the chewing gum exhibiting an antibacterial activity was taken to be “o”, and the chewing gum not exhibiting an antibacterial activity was taken to be “x”. Tables 2 to 4 show the results.

Tables 2 to 4 indicates that appropriate minimum compounding concentrations of the macrocarpal A, B, and C are 0.001% by weight, 0.003% by weight, and 0.0015% by weight, respectively.

### Example 5

Eucalyptus has distinct odor, and hence increasing the amount of Eucalyptus adversely affects taste. Further, the chewing gum surface is colored, so that appearance becomes worse. On the other hand, also from the viewpoint of physical properties, drying of the chewing gum becomes difficult, resulting in numerous chewing gums having defects in shape. In addition, storage stability is adversely affected due to moisture absorption even after production. In order to evaluate those affects, appearance evaluation and sensory evaluation were performed for a chewing gum immediately after production and a chewing gum after two-year storage at room temperature. In addition, storage stability after two years was evaluated according to a storage deterioration test method described below. Tables 2 to 4 show the results.

(Appearance Evaluation)

The color, shape, and the like were comprehensively evaluated by visual observation in terms of whether they are good or not as a chewing gum. The good case was represented by “o”, and the poor case was represented by “x”.

(Sensory Evaluation Test)

Four panels specializing in a chewing gum masticated the chewing gum 80 times per minute for consecutive 5 minutes, and sensuously evaluated masticatory comfort, taste, and the like. It should be noted that there was no comparative chewing gum at this time, and hence the chewing gum was absolutely evaluated in terms of whether it was good or not as a chewing gum. The good case was represented by “o”, and the poor case was represented by “x”.

(Storage Deterioration Test)

A chewing gum after two-year storage at room temperature was measured for its hardness with a rheometer. The hardness was taken to be an index of quality deterioration. The product exhibiting ±20% or more of change compared with the product immediately after production was judged to be poor in quality.

Rheometer: Sun Scientific Co., Ltd., RUD-J type

Pressure-sensitive axis: diameter of 3 mm

Loading range: 10 kg

Table speed: 40 mm/min

Sample pretreatment: 24 hours before measurement, a chewing gum was placed in a thermostatic chamber at 20° C., and the product temperature was kept constant.

Tables 2 to 4 indicate that the appropriate maximum compounding concentrations of the macrocarpal A, B, and C are 0.0045% by weight, 0.0045% by weight, 0.0045% by weight, respectively.

TABLE 2
Evaluation on chewing gum A

| | Concen- | | | | | Sensory evaluation and physical properties evaluation | | | | |
|---|---|---|---|---|---|---|---|---|---|---|
| | tration in | Antibacterial | | | | Immediately after | | | | |
| Content in | saliva | activity | | | | production | | After two-year storage | | |
| gum (%) | (μg/ml) | a | b | c | d | Appearance | Sense | Appearance | Sense | Hardness |
| 0.00050 | 0.66 | ∘ | ∘ | x | ∘ | ∘ | ∘ | ∘ | ∘ | Good |
| 0.00097 | 1.26 | ∘ | ∘ | ∘ | ∘ | ∘ | ∘ | ∘ | ∘ | Good |
| 0.00150 | 2.06 | ∘ | ∘ | ∘ | ∘ | ∘ | ∘ | ∘ | ∘ | Good |
| 0.00347 | 4.64 | ∘ | ∘ | ∘ | ∘ | ∘ | ∘ | ∘ | ∘ | Good |
| 0.00460 | 6.30 | ∘ | ∘ | ∘ | ∘ | ∘ | ∘ | ∘ | ∘ | Good |
| 0.00495 | 6.76 | ∘ | ∘ | ∘ | ∘ | ∘ | ∘ | x | ∘ | Poor |
| 0.00552 | 7.30 | ∘ | ∘ | ∘ | ∘ | x | x | x | x | Poor |

TABLE 3
Evaluation on chewing gum B

| | Concen- | | | | | Sensory evaluation | | | | |
|---|---|---|---|---|---|---|---|---|---|---|
| | tration in | Antibacterial | | | | Immediately after | | | | |
| Content in | saliva | activity | | | | production | | After two-year storage | | |
| gum (%) | (μg/ml) | a | b | c | d | Appearance | Sense | Appearance | Sense | Hardness |
| 0.00120 | 1.60 | ∘ | ∘ | x | x | ∘ | ∘ | ∘ | ∘ | Good |
| 0.00178 | 2.20 | ∘ | ∘ | x | ∘ | ∘ | ∘ | ∘ | ∘ | Good |
| 0.00287 | 3.43 | ∘ | ∘ | ∘ | ∘ | ∘ | ∘ | ∘ | ∘ | Good |
| 0.00450 | 5.45 | ∘ | ∘ | ∘ | ∘ | ∘ | ∘ | ∘ | ∘ | Good |
| 0.00495 | 6.02 | ∘ | ∘ | ∘ | ∘ | ∘ | x | ∘ | x | Good |
| 0.00556 | 6.55 | ∘ | ∘ | ∘ | ∘ | ∘ | x | ∘ | x | Poor |

TABLE 4
Evaluation on chewing gum C

| | Concen- | | | | | Sensory evaluation | | | | |
|---|---|---|---|---|---|---|---|---|---|---|
| | tration in | Antibacterial | | | | Immediately after | | | | |
| Content in | saliva | activity | | | | production | | After two-year storage | | |
| gum (%) | (μg/ml) | a | b | c | d | Appearance | Sense | Appearance | Sense | Hardness |
| 0.00090 | 1.30 | ∘ | ∘ | x | ∘ | ∘ | ∘ | ∘ | ∘ | Good |
| 0.00147 | 1.96 | ∘ | ∘ | ∘ | ∘ | ∘ | ∘ | ∘ | ∘ | Good |
| 0.00205 | 2.87 | ∘ | ∘ | ∘ | ∘ | ∘ | ∘ | ∘ | ∘ | Good |
| 0.00408 | 5.44 | ∘ | ∘ | ∘ | ∘ | ∘ | ∘ | ∘ | ∘ | Good |
| 0.00450 | 5.78 | ∘ | ∘ | ∘ | ∘ | ∘ | ∘ | ∘ | ∘ | Good |
| 0.00495 | 6.45 | ∘ | ∘ | ∘ | ∘ | x | x | x | x | Poor |
| 0.0059 | 7.45 | ∘ | ∘ | ∘ | ∘ | x | x | x | x | Poor |

This application claims the benefit of Japanese Patent Application Number 2008-052267 filed on Mar. 3, 2008, which is hereby incorporated by reference herein in its entirety.

1. A chewing gum containing Eucalyptus extract, comprising macrocarpal A in an amount of 0.001 to 0.0045% by weight with respect to a total weight of the chewing gum.

2. A chewing gum containing Eucalyptus extract, comprising macrocarpal B in an amount of 0.003 to 0.0045% by weight with respect to a total weight of the chewing gum.

3. A chewing gum containing Eucalyptus extract, comprising macrocarpal C in an amount of 0.0015 to 0.0045% by weight with respect to a total weight of the chewing gum.

4. A chewing gum containing Eucalyptus extract, comprising at least two kinds of macrocarpal A, macrocarpal B, and macrocarpal C, contents of the macrocarpal A, the macrocarpal B, and the macrocarpal C being 0.001 to 0.0045% by weight, 0.003 to 0.0045% by weight, and 0.0015 to 0.0045% by weight with respect to a total weight of the chewing gum, respectively.
